(19) United States
(12) Patent Application Publication
(10) Pub. No.: US 2018/0110972 A1
(43) Pub. Date: Apr. 26, 2018

(54) SYSTEMS AND METHODS FOR INTRACRANIAL DEVICE IMPLANTATION

(71) Applicant: THE CLEVELAND CLINIC FOUNDATION, Cleveland, OH (US)

(72) Inventors: Jorge Gonzalez-Martinez, University Heights, OH (US); John T. Gale, Chardon, OH (US)

(21) Appl. No.: 15/790,149

(22) Filed: Oct. 23, 2017

Related U.S. Application Data
(60) Provisional application No. 62/410,898, filed on Oct. 21, 2016.

(51) Int. Cl. A61N 1/05; A61B 5/03

(52) U.S. Cl. CPC A61N 1/0534; A61B 5/6851; A61B 5/031; A61N 1/0539

(57) ABSTRACT

The present disclosure relates generally to implanting intracranial devices (e.g., electrodes, catheters, biopsy probes, etc.) independently (not in parallel) into a patient's brain. A single entry point can be opened in a patient's head. At least two intracranial devices can be inserted through the entry point. The at least two intracranial devices can be implanted to at least two different locations in the patient's brain through the same entry point.

### CROSS-REFERENCE TO RELATED APPLICATIONS

This application claims the benefit of U.S. Provisional Application No. 62/410,898, entitled “SYSTEMS AND METHODS FOR IMPLANTING INTRACRANIAL DEVICES,” filed 21 Oct. 2016. The entirety of this provisional application is hereby incorporated by reference for all purposes.

### TECHNICAL FIELD

The present disclosure relates generally to intracranial device implantation and, more specifically, to systems and methods for independent implantation of multiple intracranial devices into a patient's brain through a single entry point in the skull.

### BACKGROUND

Epilepsy is among the most common disorders of the nervous system, affecting as many as 50 million people worldwide. As many as 30% of the people with epilepsy as characterized as having medically refractory epilepsy (MRE), which is not responsive or minimally responsive to anti-epileptic drugs. In addition, many other neurological disorders continue to have poor outcomes despite management with invasive or noninvasive therapies. These include chronic pain conditions, including neuropathic pain, as well as cancer related pain, movement disorders such as Parkinson's disease, essential tremor, symptomatic tremors, and other movement problems.

A number of surgical interventions are possible for these disorders, all of which rely on the precise localization of therapeutic areas within the brain. For epileptic patients, stereoelectroencephalography (SEEG) can be used for precise localization of the therapeutic area. With SEEG, electroencephalographic signals can be recorded using depth electrodes that are surgically implanted into brain tissue. The electroencephalographic signals can be processed to localize the therapeutic areas.

### SUMMARY

The present disclosure relates generally to intracranial device (e.g., electrodes, catheters, biopsy probes, etc.) implantation. Traditionally, intracranial devices have been implanted through individual entry points into the skull. Multiple intracranial devices could only be implanted in parallel through the same entry point. The systems and methods of the present disclosure can facilitate the independent implantation of multiple intracranial devices into a patient's brain through a single entry point in the skull. Notably, the multiple intracranial devices are not required to be in parallel.

In one aspect, the present disclosure includes a method for implanting multiple intracranial devices into a patient's brain through a single entry point. An entry point can be opened in a patient's head. At least two intracranial devices can be inserted through the entry point. The at least two intracranial devices can be implanted to at least two different locations in the patient's brain.

In another aspect, the present disclosure includes a system for implanting multiple intracranial devices into the patient's brain through a single entry point. The system includes at least two intracranial devices implantable to different locations through the same entry point in a patient's head, The system also includes a steerable guide probe to guide the at least two intracranial devices along at least two predefined trajectories to the different locations. The steerable guide probe, in some instances, can exit from the brain after implantation of one of the implantable devices so that the steerable guide can guide another implantable device to another location. The system also includes a fluoroscopic imaging modality to track the steerable guide probe as it travels along the at least two predefined trajectories to the different locations.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is diagram of a system that can be used for implanting multiple intracranial devices into a patient's brain through a single entry point in accordance with an aspect of the present disclosure;

FIG. 2 is an example of a control system that can be used with the system in FIG. 1;

FIGS. 3-4 are diagrams showing the implantation of multiple intracranial devices into the patient's brain through a single entry point that can be accomplished by the system in FIG. 1;

FIGS. 5-7 are process flow diagrams illustrating methods for implanting multiple intracranial devices into a patient's brain through a single entry point in accordance with another aspect of the present disclosure.

### DETAILED DESCRIPTION

### I. Definitions

In the context of the present disclosure, the singular forms “a,” “an” and “the” can also include the plural forms, unless the context clearly indicates otherwise.

The terms “comprises” and/or “comprising,” as used herein, can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “intracranial device” can refer to an instrument, apparatus, implant, or similar related article that is used to diagnose, prevent, or treat disease or other conditions within the skull. Examples of intracranial devices include electrodes, catheters, biopsy probes, and the like.

As used herein, the term “guide probe” can refer to a medical instrument used for placement of another device in the brain. In some instances, the other device can be larger than the guide probe. Examples of the guide probe can include a guide wire/spring, an inflatable balloon, or the like.

As used herein, the term “independently” can refer to two or more intracranial devices being implanted into a patient's brain through the same burr hole without the intracranial devices being in parallel.

As used herein, the term “fluoroscopic imaging modality” can refer to an imaging technique that allows a medical professional to visualize the internal structure and/or function of a patient. In some instances, the fluoroscopic imaging modality can be utilized during a surgical procedure (e.g., to guide the placement of intracranial devices within the brain). One example of a fluoroscopy imaging modality can include live biplane fluoroscopic imaging. However, other imaging modalities can also be used as a live fluoroscopic imaging modality, including computed tomography, magnetic resonance imaging, other types of fluoroscopy, etc.

As used herein, the term “stereotactic” can refer to a technique for locating one or more points inside a patient's brain using an external, three-dimensional frame of reference (real or virtual) based on a three-dimensional coordinate system.

As used herein, the term “medical professional” can refer to can refer to any person involved in medical care of a patient including, but not limited to, physicians, medical students, nurse practitioners, nurses, and technicians.

As used herein, the term “patient” can refer to any warm-blooded organism including, but not limited to, a human being, a pig, a rat, a mouse, a dog, a cat, a goat, a sheep, a horse, a monkey, an ape, a rabbit, a cow, etc. The terms “patient” and “subject” can be used interchangeably herein.

As used herein, the term “neurological condition” can refer to a disorder of the nervous system resulting from an abnormality (e.g., biochemical, structural, and/or electrical). Examples of neurological conditions can include epilepsy, movement disorder, chronic pain, cancer, a psychiatric disorder, or the like.

As used herein, the term “real time” can refer to the actual time during which a process or event occurs. In other words, an image that is recorded in real time can be processed within milliseconds so that it is available virtually immediately as feedback.

### II. Overview

The present disclosure relates generally to implanting intracranial devices into a patient's brain. Traditionally, a plurality of intracranial devices have been stereotactically implanted into respective locations in a patient's brain through a corresponding plurality of burr holes in the patient's skull. The same burr hole has been used to implant two or more multiple intracranial devices, as long as the multiple intracranial devices are implanted in parallel. The present disclose provides a true departure over these implantation techniques by implanting multiple intracranial devices into the patient's brain independently (not in parallel) through a single entry point (or burr hole) in the skull. The independent implantation of the multiple intracranial devices through the single entry point can be guided using a steerable guide probe (e.g., steerable guide wires, inflated balloons, or the like) that can modify the direction of the intracranial devices without injuring the brain. Using this minimally invasive approach, the implantable devices can be safely placed in specific brain regions with advantages related to time, morbidity, cost, and precision.

### III. Systems

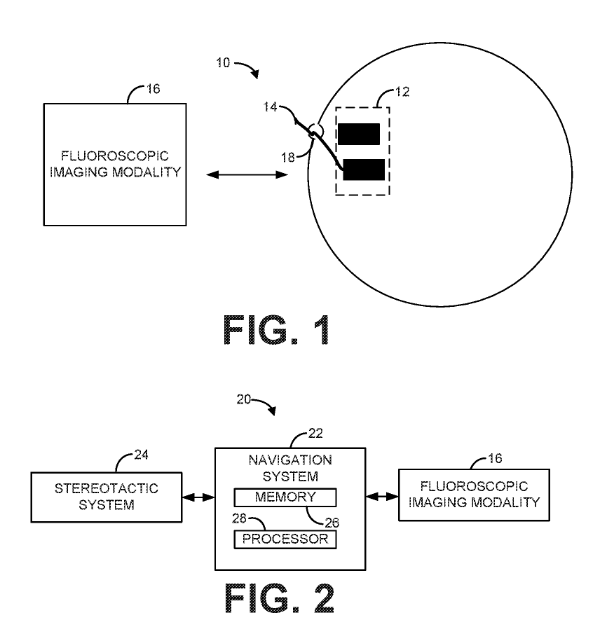

One aspect of the present disclosure can include a minimally invasive implantation system 10, as shown in FIG. 1. The minimally invasive approach employed by the system 10 can achieve advantages related to time of implantation, morbidity, costs, and precision. The implantation system 10 can include at least two intracranial devices 12, a steerable guide probe 14, and a fluoroscopic imaging modality 16.

The at least two intracranial devices 12 are independently implantable to different locations in a patient's brain through a single entry point 18 in the patient's head/skull. The intracranial devices 12 can be used for diagnosis or treatment of a neurological condition, such as epilepsy, a psychiatric disorder, movement disorder, Parkinson's disease, chronic pain, cancer, or the like. For example, the at least two intracranial devices 12 can be one or more of electrodes, catheters, brain biopsy probes, and the like.

The steerable guide probe 14 can be used to guide the at least two intracranial devices 12 along at least two predefined trajectories to the different locations. The steerable guide probe 14 can sequentially guide each of the at least two intracranial devices 12 to their unique location (entering and exiting the brain with each implantation). The steerable guide probe 14 can be, for example, a guide wire and/or an inflatable balloon. The fluoroscopic imaging modality 16 can track the steerable guide probe 14 as it travels through the patient's head along each of the predefined trajectories to the different locations.

The at least two intracranial devices 12 can be implanted under the guidance of control system 20. The control system 20 can include the fluoroscopic imaging modality 16, a navigation system 22, and a stereotactic system 24, as shown in FIG. 2. The navigation system 22 can be implemented by a computing device that includes a non-transitory memory 26 and a processor 28. One or more instructions related to execution of the system 20 can be stored on the non-transitory memory 26 and executed by the processor 28. For example, the non-transitory memory 26 can include one or more hardware memory device. However, the non-transitory memory 26 is not a transitory signal. Similarly, the processor 28 can include one or more hardware processing units.

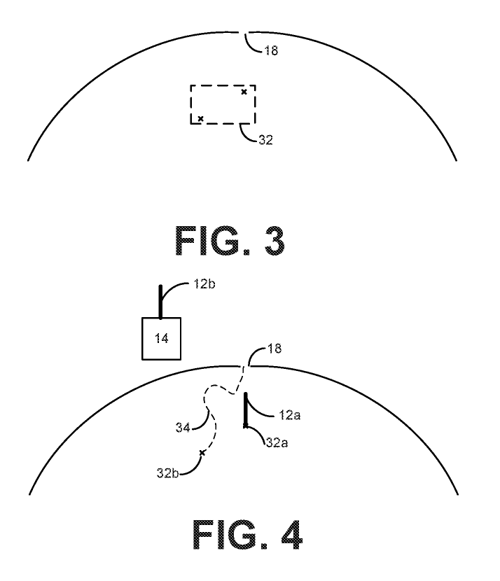

The navigation system 22 can control the implantation of the intracranial devices 12 into the patient's brain. The navigation system 22 can determine the locations for the at least two intracranial devices. The locations 32, in some instances, can be determined by the processor 28 of the navigation system 22 based on information stored in the non-transitory memory 26. The information stored in the non-transitory memory 26 can include a preimplantation image of at least a portion of the patient's brain (e.g., CT, MRI, angiogram, etc.) and/or a preimplantation electroencephalogram study (or other preimplantation study). For example, the processor 28 of the navigation system 22 can determine the locations 32 according to an algorithm (at least part of which is store in the non-transitory memory 26). The algorithm can consider a calculate probability of localization of the brain malady with a maximization of ease of access and a minimization of tissue damage when determining the locations. An example of two locations 32 determined by the navigation system 22 are shown in FIG. 3. In some instances, the locations 32 can be determined to be at different lateral positions and/or different depths within the brain.

After the locations 32 are determined, the navigation system 22 can determine the physical location of the single entry point 18 based on data stored in the memory 26 (e.g., the preoperative imaging data). For example, the location of the single entry point 20 can be based on an ease of the intracranial devices reaching the locations 18 without damaging the brain tissue. Another consideration related to the physical location of the single entry point 20 may include the external morphological features of the patient's head.

The navigation system 22 can thereafter determine coordinates of the single entry point and coordinates of the locations 32. The coordinates can be used by the stereotactic system 24 to facilitate precise implantation of the intracranial devices 12 to the locations 32. For example, the coordinates can be determined according to a map used by the stereotactic system 24. The stereotactic system 24 can include a stereotactic device that is attachable to the patient's head to facilitate the implantation of the intracranial devices 12 to the locations 32. The stereotactic device can ensure that the implantation is conducted in a consistent manner with a map and coordinates on the map corresponding to the locations. In some examples, the stereotactic devices can be a hardware stereotactic frame that is attachable to the patient's head preoperatively. In other examples, the stereotactic device can be a frameless stereotactic device.

After determining the locations 32 and the single entry point 18, the navigation system 22 can determine trajectories for the implantation of the intracranial devices 12 to the locations 32. The trajectories can be determined based on ease of implantation and/or based on biological features of the brain. The trajectories, in some examples, can be determined to cause minimal (or no) damage to vital structures within the brain. The navigation system 22 can feed the locations and the trajectories to the stereotactic system 24, which can facilitate the implantation of the intracranial devices along the trajectories. In turn, the stereotactic system 24 can feed information about the implantation to the navigation system 22. Additionally, the single entry point can be opened in the patient's skull by a component, such as a bone drill. In some instances, the component can include a skin coagulating agent and/or a dura opening component to open a portion of the dura protecting the patient's brain.

FIG. 4 shows an example of a trajectory 34 that a single intracranial device 12b can follow to reach the certain location 32b. The other intracranial device 12a has already been implanted to its location 32a. The stereotactic device of the stereotactic system 24 can be set to deliver the intracranial device 12b from the single entry point 18 to the location 32b along the predefined trajectory 34. The intracranial device 12b can be guided to the location 32b along the trajectory 34 using a steerable guide device 14.

The navigation system 22 can track the steerable guide device 14 through the patient's head based on a fluoroscopic image taken by the fluoroscopic imaging modality 16. In some instances, the fluoroscopic imaging modality 16 can provide an image during surgery in which the intracranial devices 12 are implanted into the patient's brain. The image can be updated in real time. One example of a real time imaging modality can record biplane fluoroscopic images during surgery to track the location of the steerable guide probe. The fluoroscopic imaging modality 16 can feed the real time image to the navigation system 22, which can determine whether the steerable guide probe and/or the trajectory needs to be corrected based on the real time image. In the event that a correction is needed, the navigation system 22 sends instructions to the stereotactic system 24, which makes the appropriate corrections. In other words, the stereotactic system 24, the navigation system 22, and the fluoroscopic imaging modality 16 can constitute a feedback loop, which can ensure that the intracranial devices 12 are delivered to the locations through the single entry point 18 without causing damage to vital brain structures.

### IV. Methods

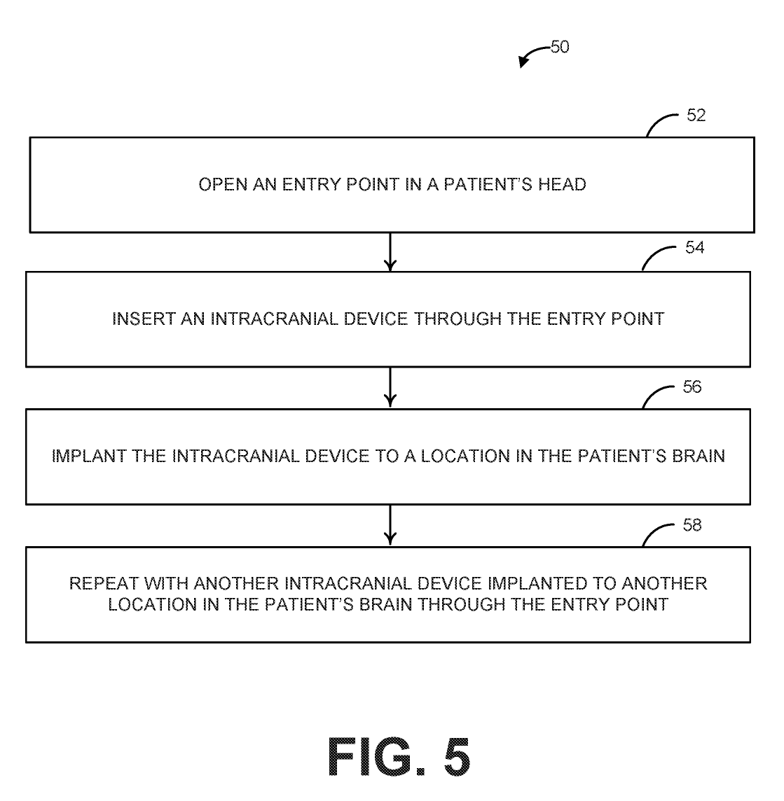

Another aspect of the present disclosure can include methods 50, 60 and 70 for implanting multiple (at least two) intracranial devices into a patient's brain through a single entry point independently (not in parallel), as shown in FIGS. 5, 6 and 7. As an example, the methods 50, 60 and 70 can be accomplished or facilitated using the systems 10 and 20 as shown in FIGS. 1 and 2. For example, the navigation system 22 can include a non-transitory memory 26 storing instructions and a processor 28 that accesses the non-transitory memory 26 and executes the instructions to perform actions, which can be one or more actions of methods 50, 60, and/or 70. It should be noted that the navigation system 22, the stereotactic system 24, and the fluoroscopic imaging modality 16 can work together as a feedback look to control the implantation of the multiple locations in the brain without damaging vital brain structures.

The methods 50, 60 and 70 are illustrated as process flow diagrams with flowchart illustrations. For purposes of simplicity, the methods 50, 60 and 70 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 50, 60 and 70.

FIG. 5 illustrates a method 50 for implanting multiple intracranial devices into the patient's brain independently (not in parallel) to different locations through a single entry point. For example, the intracranial devices can be electrodes, catheters, biopsy probes, or the like. The intracranial devices can be used for diagnosis or treatment of a neurological condition (e.g., epilepsy, a psychiatric disorder, movement disorder, Parkinson's disease, chronic pain, cancer, or the like).

At 52, the entry point can be opened in the patient's head. The entry point can be determined based on a preimplantation image (e.g., CT, MRI, angiogram, etc.) or preimplantation electroencephalogram study. Results of the preimplantation image or the preimplantation electroencephalogram study can be stored in a non-transitory memory. For example, the entry point can be determined by a processor so that the trajectories to the locations for the intracranial devices are simple or unlikely to damage neural structures. Coordinates for the entry point can be set in a stereotactic devices (e.g., frame or frameless) and can be opened exactly at the coordinates.

At 54, one of the intracranial devices can be inserted into a patient's brain through the entry point. The insertion can be conducted under the guidance of a stereotactic device. At 56, the intracranial device can be implanted to a first location in the patient's brain. The implantation can be along a trajectory defined for the intracranial device. The implantation can be guided and controlled by the stereotactic device. At 58, another intracranial device can be implanted to another location in the patient's brain through the entry point. This implantation of the second intracranial device can be conducted by repeating steps 54 and 56 according to another trajectory. The implantation of the two intracranial devices can be conducted independent of each other implantation.

Indeed, the multiple intracranial devices can be implanted independently to different predetermined locations and/or at different predefined depths in the patient's brain. Coordinates for the at least two different locations can be entered into the stereotactic device to ensure that the implantation is exact. Different trajectories can be followed so that the at least two intracranial devices can be implanted at the at least two different locations. The real time image can be used as feedback to determine whether the steerable guide probe and/or the trajectory needs to be corrected, and the correction can be made.

The at least two intracranial devices can be inserted in sequence with a steerable guide probe. The steerable guide probe can be, for example, a guide wire and/or an inflatable balloon, which can be tracked by an intraoperative imaging modality. The intraoperative imaging modality can be, for example, a fluoroscopic imaging modality that can record live (real time) biplane images that can track the steerable guide probe as it travels through the patient's brain to the location.

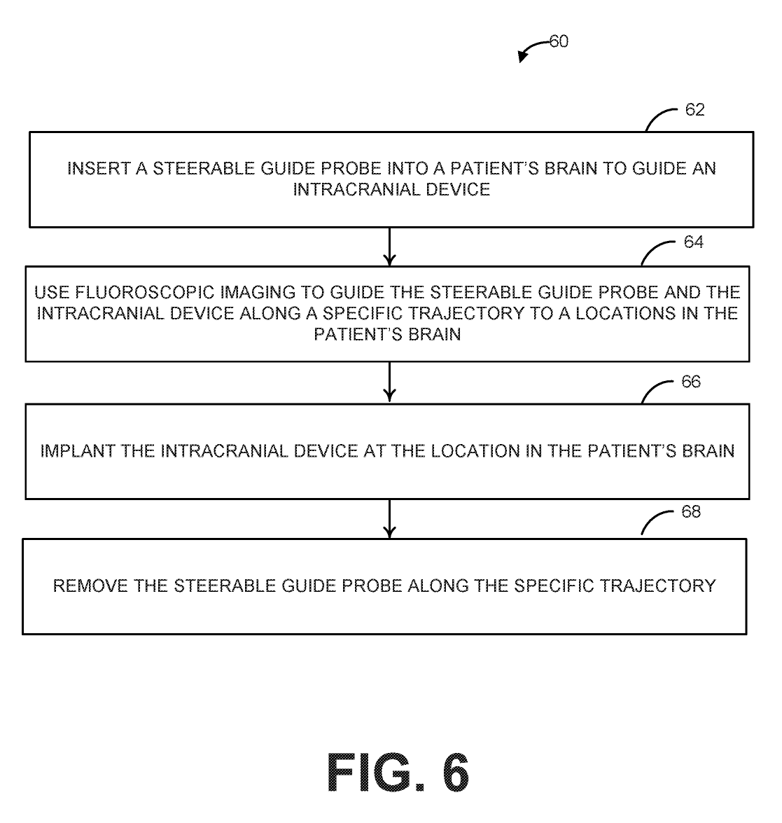

The steerable guide probe can be used to guide the at least two intracranial devices to the at least two different locations in the patient's brain. FIG. 6 illustrates a method 60 for guiding the implantation of at least two intracranial devices to at least two different locations in the patient's brain using the steerable guide probe.

At 62, the steerable guide probe can be inserted into the patient's brain. For example, the steerable guide probe can be inserted with the intracranial device to guide the intracranial device from the entry point to the location. In some instances, the steerable guide probe can be trackable by a live fluoroscopic image. For example, the steerable guide probe can be a guide wire, an inflatable balloon, or the like.

At 64, fluoroscopic imaging can be used to guide the steerable guide probe along a specific trajectory to the location for implantation of the intracranial device. For example, the fluoroscopic imaging can record a live biplane image during the implantation. The image can provide feedback related to the implantation to the location and/or the trajectory. The stereotactic device can alter the trajectory and/or the location based on the image. At 66, the intracranial device can be implanted at the location in the patient's brain. After implantation, at 68, the steerable guide device can be removed along the specific trajectory. The removal can be tracked by the love fluoroscopic image. Similarly, the stereotactic device can adjust the trajectory of removal based on the fluoroscopic image. Notably, the trajectory used to implant another intracranial device would be different from the trajectory used to implant the intracranial device.

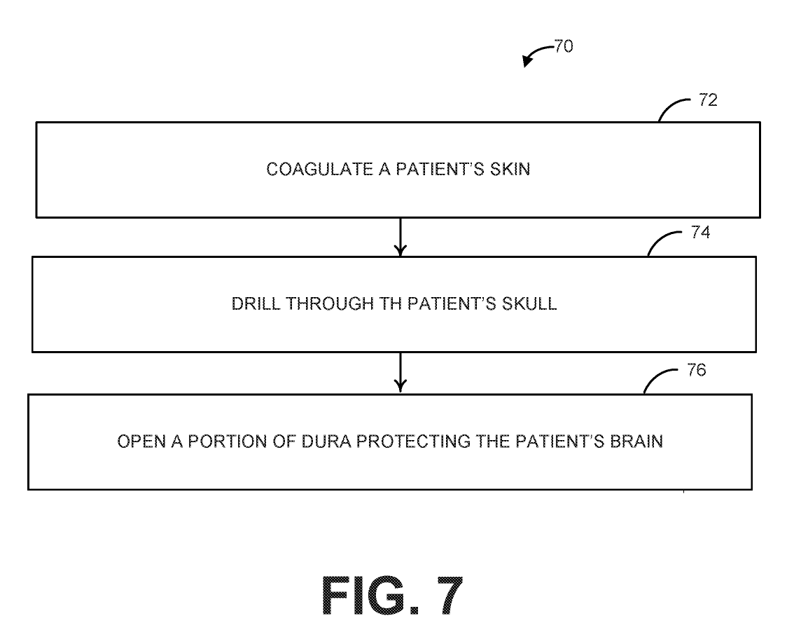

The implantation of the multiple intracranial devices can be accomplished using a percutaneous minimally invasive approach. The implantation begins with opening the entry point using a stereotactic device (frame or frameless), which is applied before the implantation. One example method 70 for creating the opening of the entry point is shown in FIG. 7.

At 72, coagulation of the patient's skin can be accomplished using one or more specific skin coagulators. At 74, the stereotactic device can guide bone drilling through the patient's skull. At 76, a portion of the dura protecting the patient's brain can be opened using a specific dura probe, guided by the stereotactic device. Upon opening the dura, the steerable guide probe can be inserted under live fluoroscopy and can travel along the specific trajectory. The intracranial device can follow the steerable guide probe to its final location.

From the above description, those skilled in the art will perceive improvements, changes and modifications. Such improvements, changes and modifications are within the skill of one in the art and are intended to be covered by the appended claims.

What is claimed is:

1. A method comprising:
opening an entry point in a patient's head;
inserting at least two intracranial devices through the entry point independently; and
implanting the at least two intracranial devices to at least two different locations in the patient's brain.

2. The method of claim 1, wherein the implanting further comprises guiding the at least two intracranial devices to the at least two different locations by a steerable guide probe.

3. The method of claim 2, further comprising viewing the steerable guide probe in the patient's brain using live fluoroscopic image guidance.

4. The method of claim 3, wherein the live fluoroscopic guidance is provided by a biplane fluoroscopic image used during surgery.

5. The method of claim 2, wherein the steerable guide probe follows a specific trajectory to each of the at least two different locations.

6. The method of claim 2, wherein the steerable guide probe is a guide wire or an inflatable balloon.

7. The method of claim 1, further comprising determining, by a system comprising a processor, a location for the entry point based on a preoperative image of the patient's brain.

8. The method of claim 7, further comprising using a stereotactic device to open the entry point precisely at the location.

9. The method of claim 1, wherein the opening the entry point further comprises:
coagulating the patient's skin;
drilling through the patient's skull; and
opening a portion of dura protecting the patient's brain.

10. The method of claim 1, wherein the intracranial devices are at least one of electrodes, catheters, and brain biopsy probes.

11. A system comprising:
at least two intracranial devices independently implantable to different locations through the same entry point in a patient's head;
a steerable guide probe to guide the at least two intracranial devices along at least two predefined trajectories to the different locations; and
a fluoroscopic imaging modality to track the steerable guide probe as it travels through the patient's head along the at least two predefined trajectories to the different locations.

12. The system of claim 11, further comprising a stereotactic device attachable to the patient's head to facilitate the implantation of the at least two intracranial devices and the steerable guide probe.

13. The system of claim 12, wherein the implantation of the at least two intracranial device using the stereotactic device is controlled by a navigation system comprising a non-transitory memory and a processor.

14. The system of claim 13, wherein the navigation system is configured to track the steerable guide device through the patient's head based on a fluoroscopic image taken by the fluoroscopic imaging modality.

15. The system of claim 11, wherein the intracranial devices are at least one of electrodes, catheters, and brain biopsy probes.

16. The system of claim 11, wherein the steerable guide probe is a guide wire or an inflatable balloon.

17. The system of claim 11, further comprising a component to open the entry point in the patient's skull.

18. The system of claim 17, wherein the component to open the entry point in the patient's skill comprises a bone drill.

19. The system of claim 17, wherein the component to open the entry point in the patient's skull comprises a skin coagulating agent.

20. The system of claim 18, wherein the component to open the entry point in the patient's skull comprises a dura opening component to open a portion of the dura protecting the patient's brain.
